(19) United States
(12) Patent Application Publication
(10) Pub. No.: US 2018/0346966 A1
(43) Pub. Date: Dec. 6, 2018

(54) PRIMER COMPOSITIONS AND MULTIPLEX METHODS FOR DETECTING THE PRESENCE OF LISTERIA

(71) Applicant: Conagra Foods RDM, Inc., Omaha, NE (US)

(72) Inventors: Bradley Ziebell, Omaha, NE (US); Kari Sweeney, Omaha, NE (US)

(21) Appl. No.: 15/941,685

(22) Filed: Mar. 30, 2018

Related U.S. Application Data
(60) Provisional application No. 62/479,695, filed on Mar. 31, 2017.
(60) Provisional application No. 62/526,584, filed on Jun. 29, 2017.

(51) Int. Cl. C12Q 1/689

(52) U.S. Cl. CPC C12Q 1/689; C12Q 1/686; C12Q 2600/16

(57) ABSTRACT

Aspects of the disclosure relate to primer compositions and multiplex methods for detecting the presence of Listeria. The primer composition includes a first primer sequence operable to amplify a first polymerase chain reaction (PCR) product (e.g., a nucleic acid, a fragment thereof, a complement thereof, or a gene). The first primer sequence is specific for the Listeria genus. The primer composition also includes at least one additional primer sequence operable to amplify at least one additional PCR product. The additional primer sequence(s) are specific for one or more species or subspecies of Listeria. The multiplex method includes contacting a sample with the primer composition to substantially simultaneously detect the presence of the Listeria genus and differentiate between species of Listeria present in the sample.

### CROSS-REFERENCE TO RELATED APPLICATIONS

The present application claims the benefit under 35 U.S.C. § 119(e) of U.S. Provisional Application Ser. No. 62/479,695, filed Mar. 31, 2017, and titled “PRIMER COMPOSITION AND MULTIPLEX METHOD FOR DETECTING THE PRESENCE OF LISTERIA;” and U.S. Provisional Application Ser. No. 62/526,584, filed Jun. 29, 2017, and titled “PRIMER COMPOSITIONS AND MULTIPLEX METHODS FOR DETECTING THE PRESENCE OF LISTERIA.” U.S. Provisional Application Ser. Nos. 62/479,695 and 62/526,584 are herein incorporated by reference in their entireties.

### INCORPORATION OF SEQUENCE LISTING

The sequence listing contained in the file named “SeqListing08-21-2018_ST25.txt”, which is 2.21 kilobytes in size (measured in MS-Windows®), contains 13 sequences, and which was created on Aug. 21, 2018, is contemporaneously filed with this specification by electronic submission (using the United States Patent Office EFS-Web filing system) and is incorporated herein by reference in its entirety.

### BACKGROUND

Listeria is a genus of bacteria. Some species of Listeria can cause the food-borne disease, Listeriosis, which has a mortality rate of 20 to 30% in humans.

### SUMMARY

This Summary is provided to introduce a selection of concepts in a simplified form that are further described below in the Detailed Description. This Summary is not intended to identify key and/or essential features of the claimed subject matter. Also, this Summary is not intended to limit the scope of the claimed subject matter in any manner.

Aspects of the disclosure relate to primer compositions and multiplex methods for detecting the presence of Listeria. The primer composition includes a first primer sequence operable to amplify a first polymerase chain reaction (PCR) product (e.g., a nucleic acid, a fragment thereof, a complement thereof, or a gene). The first primer sequence is specific for the Listeria genus. The primer composition also includes at least one additional primer sequence operable to amplify at least one additional PCR product. The additional primer sequence(s) are specific for one or more species or subspecies of Listeria. The multiplex method includes contacting a sample with the primer composition to substantially simultaneously detect the presence of the Listeria genus and differentiate between species of Listeria present in the sample.

In some embodiments, the primer composition includes a first primer set and at least one additional primer set. The first primer set is operable to amplify a first PCR product. The first primer set includes at least a first primer sequence and a second primer sequence that are specific for genus Listeria. The additional primer set(s) are operable to amplify one or more additional PCR products. The additional primer set includes at least a third primer sequence and a fourth primer sequence specific for at least one of a species of Listeria or a subspecies thereof.

In some embodiments, the first primer set includes at least one of a first set of sequences selected from SEQ ID NO: 12 and SEQ ID NO: 13, a set of nucleic acid molecules with at least 90% sequence homology to the first set of sequences, a set of nucleic acid molecules complementary to the first set of sequences, or a set of nucleic acid molecules complementary to the nucleic acid molecules with at least 90% sequence homology to the first set of sequences. The additional primer set(s) includes at least one of a second set of sequences selected from at least one of SEQ ID NO: 1 and SEQ ID NO: 2, SEQ ID NO: 3 through SEQ ID NO: 5, SEQ ID NO: 6 and SEQ ID NO: 7, SEQ ID NO: 8 and SEQ ID NO: 9, or SEQ ID NO: 10 and SEQ ID NO: 11, a set of nucleic acid molecules with at least 90% sequence homology to the second set of sequences, a set of nucleic acid molecules complementary to the second set of sequences, or a set of nucleic acid molecules complementary to the nucleic acid molecules with at least 90% sequence homology to the second set of sequences.

In some embodiments, the method for determining the presence of Listeria organism in a sample includes contacting the sample with a primer composition. The primer composition includes a first primer set operable to amplify a first PCR product and at least one additional primer set operable to amplify at least a second PCR product. The method further includes detecting the first PCR product and the second PCR product. Detecting the first PCR product is indicative of the presence of genus Listeria in the sample, and detecting the second PCR product, is indicative of at least one species of Listeria or a subspecies thereof.

### DRAWINGS

The patent or application file contains at least one drawing executed in color. Copies of this patent or patent application publication with color drawing(s) will be provided by the Office upon request and payment of the necessary fee.

The Detailed Description is described with reference to the accompanying figures. The use of the same reference numbers in different instances in the description and the figures may indicate similar or identical items.

FIG. 1 is an example table illustrating primers present in a primer composition for Listeria species-specific and genus identification in accordance with example embodiments of the present disclosure.

FIG. 2 is an example table illustrating Listeria species cultures used for development of primer compositions and multiplex methods in accordance with example embodiments of the present disclosure.

FIG. 3 is an example table illustrating non-Listeria species cultures used for development of primer compositions and multiplex methods in accordance with example embodiments of the present disclosure.

FIG. 4 is an example table illustrating a multiplex PCR cycling program in accordance with example embodiments of the present disclosure.

FIG. 5 is an agarose gel assay illustrating the approximate sizes of both species and genus PCR products for each Listeria species ATCC strain during optimization of the multiplex PCR method in accordance with example embodiments of the present disclosure.

FIG. 6 is an agarose gel assay illustrating that no PCR products were substantially amplified by the multiplex PCR method after testing non-Listeria strains in accordance with example embodiments of the present disclosure.

FIG. 7A is an agarose gel assays illustrating both the species and genus PCR products for 64 unknown Listeria species isolates amplified by the multiplex PCR method after completing verification testing in accordance with example embodiments of the present disclosure.

FIG. 7B is another agarose gel assays illustrating both the species and genus PCR products for 64 unknown Listeria species isolates amplified by the multiplex PCR method after completing verification testing in accordance with example embodiments of the present disclosure.

FIG. 7C is another agarose gel assays illustrating both the species and genus PCR products for 64 unknown Listeria species isolates amplified by the multiplex PCR method after completing verification testing in accordance with example embodiments of the present disclosure.

FIG. 7D is another agarose gel assays illustrating both the species and genus PCR products for 64 unknown Listeria species isolates amplified by the multiplex PCR method after completing verification testing in accordance with example embodiments of the present disclosure.

### DETAILED DESCRIPTION

L. monocytogenes, L. innocua, L. ivanovii, L. seeligeri, L. welshimeri, and L. grayi are the most widely studied of the 17 species within the genus of Listeria. However, L. monocytogenes is the only species that has been associated with human illness. In the food industry, detection of Listeria can help prevent the spread of food-borne illness.

Aspects of the disclosure relate to primer compositions and multiplex methods for detecting the presence of Listeria. The primer composition includes a first primer sequence operable to amplify a first polymerase chain reaction (PCR) product e.g., a nucleic acid, a fragment thereof, or a complement thereof). The first primer sequence is specific for a first species of Listeria and/or the Listeria genus. The primer composition also includes at least one additional primer sequence operable to amplify at least one additional PCR product. The additional primer sequence(s) are specific for one or more additional species of Listeria.

Multiplex methods include contacting a sample with the primer composition to substantially simultaneously detect the presence of the Listeria genus and differentiate between species of Listeria present in the sample. The primer composition includes a first primer set operable to amplify a first nucleic acid, a fragment thereof, a complement thereof, or a gene. The primer composition also includes at least one additional primer set operable to amplify one or more additional nucleic acids, fragments thereof, complements thereof, or genes. For example, the primer composition includes a second primer set operable to amplify at least a second nucleic acid, fragment thereof, complement thereof, or gene. In some embodiments, the primer composition also includes at least third primer set operable to amplify at least a third nucleic acid, fragment thereof, complement thereof, or gene. The method further includes detecting at least one of the first amplified nucleic acid, a fragment thereof, a complement thereof, or a gene; the at least a second amplified nucleic acid, a fragment thereof, a complement thereof, or a gene. Detecting the first amplified nucleic acid, a fragment thereof, a complement thereof, or a gene is indicative of the presence of genus Listeria in the sample, and detecting the at least a second amplified nucleic acid, a fragment thereof, a complement thereof, or a gene is indicative of at least one of a species of Listeria or a subspecies thereof in the sample. The method can further include detecting the third amplified nucleic acid, fragment thereof, complement there or gene. Detecting the third amplified nucleic acid, fragment thereof, complement there or gene is indicative of at least a second species of Listeria or a subspecies thereof in the sample.

The multiplex methods rely on PCR techniques. Generally, a sample is lysed to release the DNA contained in organisms present in the sample. DNA primer templates are added including specific nucleotide sequences that complement known portions of genes which are unique to the target analyte organisms. A DNA polymerase and nucleotides are added, and in vitro enzymatic replication of DNA occurs. As replicate DNA strands are generated, they serve as templates for the next cycle of replication. Generally, cycles are defined by alternating heating and cooling of the sample to allow the DNA to physically separate and join, with the thermal cycling conditions providing another means of enhancing selectivity. With PCR it is possible to amplify a single or a few copies of a specific strand of DNA across several orders of magnitude, generating millions or more copies of the specific strand of DNA. Thereafter, the DNA is generally separated using agarose gel chromatography, and the DNA product, if present, is visualized by the application of a dye to the agarose gel.

The multiplex method uses multiple primers during the PCR techniques (referred as multiplex PCR) to offer high specificity and rapid (same day) Listeria species-specific identification by amplifying multiple different DNA sequences substantially simultaneously in a single test reaction. In embodiments, the multiplex PCR method can substantially simultaneously detect up to six (6) different species of Listeria, as well as the genus for Listeria. For example, the multiplex PCR method can provide substantially simultaneous differentiation between one or more of L. monocytogenes, L. innocua, L. ivanovii, L. seeligeri, L. welshimeri, L. grayi subsp. grayi, and L. grayi subsp. Murrayi. This method also confirms the presence of genus Listeria within the same PCR reaction.

### Example Multiplex PCR Primer Composition

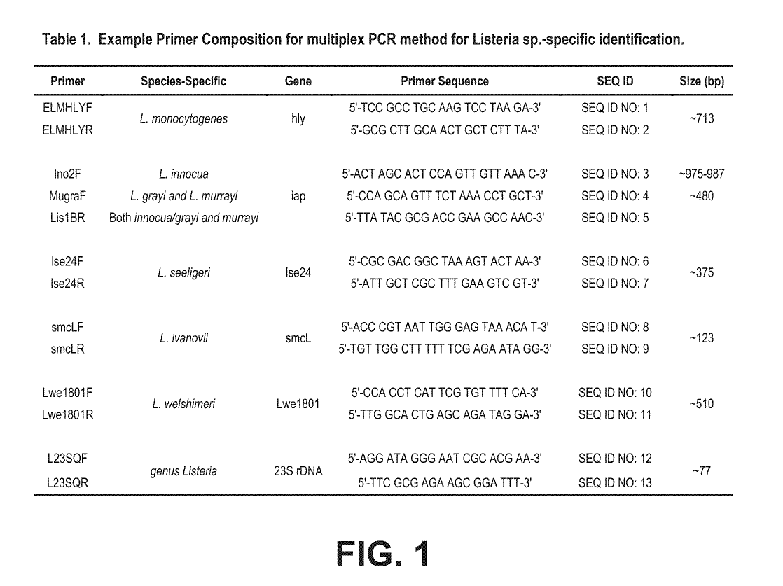

FIG. 1 illustrates several isolated strains of species and subspecies of Listeria that may include targets detectable using the multiplex PCR compositions and methods described herein. In example embodiments, target Listeria species and subspecies can include, but are not necessarily limited to: L. monocytogenes, L. innocua, L. grayi subsp. grayi, L. grayi subsp. murrayi, L. seeligeri, L. ivanovii, L. welshimeri, and so forth. The target can also include the genus Listeria.

FIG. 1 further illustrates primers for identification and detection of the Listeria target species, subspecies, and genus. In embodiments, the primer composition includes one or more of these primers. The PCR primers can be selected based on amplification of a single PCR product (e.g., a nucleic acid, a fragment thereof, or a complement thereof) at the expected size and/or absence of masking effects on the other primers included in each primer mix. Example primer pairs are described in FIG. 1 as SEQ ID NO: 1-SEQ ID NO: 13. In example embodiments, the reaction primer composition includes one or more primer pairs that may be used in assays for specific, efficient, and substantially simultaneous detection of Listeria organisms in samples. In some embodiments, the primer composition can further include one or more corresponding probes.

In some embodiments, the primer composition can include one or more duplexed sets of primer pairs and probes for detection of Listeria species, subspecies, strains, serotypes, and/or genus Listeria in a single assay. For example, the primer composition may include at least two sets of primer pairs, a first primer set including a first primer (a first forward primer) and a second primer (a first reverse primer) and a second primer set including a first primer (a second forward primer) and a second primer (a second reverse primer), each primer set operable to amplify a different species or subspecies of Listeria, or genus Listeria. For example, each primer set can be configured to amplify a different target gene or target nucleic acid fragment of the Listeria species, subspecies, or genus. In some embodiments, the primer composition may also include a corresponding probe sequence that can hybridize to amplified target nucleic acids of each primer set (a first probe and a second probe). For example, a duplexed primer set may be operable to amplify at least two different target nucleic acid sequences and their corresponding probes are operable to identify at least two different target nucleic acid sequences. In embodiments, the primer composition may include additional primer pairs (such as three primer pairs, four primer pairs and optionally the same number of corresponding probes as well), with each primer pair (and optional corresponding probe) configured to amplify a different species or subspecies of Listeria, and/or genus Listeria.

In example embodiments, the primer composition includes at least one primer configured for detecting the presence of a Listeria species or subspecies. For example, the primer composition can include at least one primer, or primer set, selected from SEQ ID NO: 1-SEQ ID NO: 11. The primer composition also includes at least one primer, or primer set configured for detecting the presence of the Listeria genus. For example, the primer composition can include at least one primer, or primer pair, selected from SEQ ID NO: 12-SEQ ID NO: 13.

In some embodiments, the primer composition includes a first primer set, the first primer set configured for detecting genus Listeria, and a second primer set configured for detecting a species or subspecies of Listeria. For example, the primer composition can include at least a first primer set, the first primer set including a first primer and a second primer, the first primer and the second primer including SEQ ID NO: 12 and SEQ ID NO: 13. The primer set(s) may also include one or more additional primers operable to amplify at least one additional PCR product. For example, the primer composition includes at least a second primer set, the second primer set including at least a third primer and a fourth primer, the third primer and the fourth primer selected from SEQ ID NO: 1-SEQ ID NO: 11. The second primer set is operable to detect a first species and/or subspecies of genus Listeria. In some embodiments, the primer composition also includes at least at least a third primer set, the third primer set including at least a fifth primer and a sixth primer, the fifth primer and the sixth primer selected from SEQ ID NO: 1 through SEQ ID NO: 11. The third primer set is operable to detect a second species and/or subspecies of genus Listeria. In a specific embodiment, the primer composition includes five or more primer sets configured for detecting different species and/or subspecies of genus Listeria, and a primer set configured for detecting genus Listeria. For example, the primer composition can include a first primer set including two primers including SEQ ID NO: 12 and SEQ ID NO: 13. The primer composition also includes a second primer set, a third primer set, a fourth primer set, a fifth primer set, and a sixth primer set, each primer set including at least two primers selected from SEQ ID NO: 1-SEQ ID NO: 11, each of the primer sets configured to detect one or more different species or subspecies of Listeria. In some embodiments, each primer set is configured to detect a specific gene corresponding to a species or subspecies of Listeria, or genus Listeria (e.g., as described with reference to FIG. 1).

### Example Multiplex PCR Methods

In some embodiments, a method may include detecting, in a sample, one or more species, subspecies, strains, serotypes, and/or the Listeria genus in a single assay. For example, the method can include detecting PCR products (e.g., nucleic acid sequence(s) having at least 10 to at least 25 nucleic acids, and/or complementary sequences thereof, or genes) wherein detection of at least one nucleic acid sequence or gene indicates the presence of a Listeria organism in the sample. In some embodiments this method may include detecting a Listeria organism of the species L. monocytogenes, L. innocua, L. grayi subsp. grayi, L. grayi subsp. murrayi, L. seeligeri, L. ivanovii, L. welshimeri, and so forth. The method may also include detecting the Listeria genus. In a specific embodiment, the method includes detecting at least one Listeria species or subspecies, and the Listeria genus, at least substantially simultaneously (e.g., using one primer composition in a single assay).

In some embodiments, a method of the disclosure may further include preparing a sample for PCR amplification, for example, but not limited to (1) bacterial enrichment, (2) separation of bacterial cells from the sample, (3) cell lysis, and/or (4) nucleic acid extraction (e.g. total DNA, genomic DNA).

Samples may include, but are not necessarily limited to: clinical samples, food samples, beverage samples, water samples, environmental samples, and so forth. Food samples may include raw produce, meats, as well as a selectively enriched food matrices.

In some embodiments, a method for the detection of a Listeria species or subspecies in a sample includes a) contacting the sample with at least a first pair of PCR primers including a forward primer and a reverse primer (e.g., selected from a row in Table 1 shown in FIG. 1) that are operable to bind to and amplify a gene or fragment thereof found only in a specific Listeria species or subspecies and not in other organisms; b) amplifying the gene or fragment thereof found only in the Listeria species or subspecies and not in other organisms to form an amplified target nucleotide sequence product; c) detecting the amplified target nucleotide sequence product; wherein the detection of the amplified target nucleotide sequence product is indicative of the presence of one or more Listeria species or subspecies in the sample. The method can also include d) contacting the sample with at least a second pair of PCR primers including a forward primer and a reverse primer (e.g., selected from a row in Table 1 shown in FIG. 1) that are operable to bind and amplify a gene or fragment thereof found only in the Listeria genus and not in other organisms; e) amplifying the gene or fragment thereof found only in the Listeria genus and not in other organisms to form at least a second amplified target nucleotide sequence product; and f) detecting the second amplified target nucleotide sequence product; wherein the detection of the second amplified target nucleotide sequence product is indicative of the presence of one or more Listeria genus in the sample.

Methods of the disclosure may include assays such as PCRs, wherein amplifying of said first pair of nucleotide primers occurs in a first vessel and said amplifying of said second pair of nucleotide primers occurs in a second vessel, or amplifying of said first pair of nucleotide primers and amplifying of said second pair of nucleotide primers occurs in a single vessel. The detection may be a real-time assay (e.g., SYBR™ Green dye assay of ThermoFisher Scientific, TAQMAN® assay of ThermoFisher Scientific, etc.).

Detection may be performed by a variety of methods, including, but not necessarily limited to: by a nucleic acid amplification reaction. The amplification reaction may be an end-point determination, the amplification reaction may be quantitative, where the quantification may be a traditional PCR (e.g., non-real-time PCR) or a real-time PCR.

In some embodiments, the detection includes an end-point determination (e.g., traditional PCR). Detection by traditional PCR methods can include subjecting the sample (after contact with the primer composition) to a series of PCR cycles at different temperatures to allow for denaturation of the sample DNA, annealing of the sample to hybridize the sample DNA to the primers, and amplification of the hybridized DNA strands by extension/elongation. The PCR cycles can be optimized to achieve efficient denaturation, hybridization, and elongation of the target Listeria genes and the primers. For example, heating the sample to a temperature in the range of approximately 85° C. to approximately 100° C. allows for denaturation of sample DNA. Further, an annealing temperature can be selected that allows the primers to efficiently hybridize with the primers. In example implementations, an annealing temperature is selected in the range of 50° C. to 65° C. The PCR cycles can be repeated multiple times to ensure substantially full hybridization and elongation. The sample can then be cooled indefinitely. Species and genus identification of the PCR product can then be determined based on the specific size (bp; migration position on a gel; e.g., as described with reference to FIG. 1) of the PCR product. For example, agarose gel analysis can be utilized to assess migration position based on size.

It is also contemplated that the detection can include real-time PRC. Real-time PCR may be a SYBR® Green Assay, and/or the real-time PCR may be a TAQMAN® Assay. Detection in some embodiments may be performed by hybridization using probes specific to target sequences. Combinations of amplification and hybridization may be used for detection according to some embodiments. For example, the method can include a first probe and a second probe, wherein the first and second probes are different from each other, the first probe operable to identify the first amplified target polynucleotide sequence and the second probe operable to identify the second amplified target nucleotide sequence. The first probe can further include a first label and said second probe can further include a second label, wherein both labels are selected from a dye, a radioactive isotope, a chemiluminescent label, and an enzyme. The dye can include a fluorescent dye, a rhodamine dye, or a cyanine dye, or a combination thereof. For example, the first probe can be labeled with a first fluorescent dye (e.g., FAM™ dye of ThermoFisher Scientific) and the second probe can be labeled with a second fluorescent dye (e.g., VIC™ dye of ThermoFisher Scientific). The first and second probes can be hybridized to the PCR amplified fragments to detect the presence of the first amplified target polynucleotide sequence and the second amplified target polynucleotide sequence from the sample.

In exemplary embodiments, a method for determining the presence of at least one Listeria species or subspecies, and the presence of the Listeria genus, in a sample includes combining the sample with a culture medium for enriching Listeria species for a time to generate a sample enriched for said species; extracting nucleic acid from at least some of the enriched sample. The method further includes contacting the extracted nucleic acid with a primer composition including at least one primer set having a specificity for amplifying one or more genes of one or more Listeria species, or a fragment of said genes, the primer set(s) having SEQ ID NO: 1 and SEQ ID NO: 2; and/or SEQ ID NO: 3, SEQ ID NO: 4, and SEQ ID NO: 5; and/or SEQ ID NO: 6 and SEQ ID NO: 7, and/or SEQ ID NO: 8 and SEQ ID NO: 9, and/or SEQ ID NO: 10 and SEQ ID NO: 11, or a nucleic acid molecule with at least 90% sequence homology to said SEQ ID sequences under conditions to generate amplified nucleic acid; and detecting at least some of the amplified nucleic acid, thereby determining the presence of one or more Listeria species or subspecies in the sample. In some embodiments, at least two of the above referenced primer sets may be used. In a specific embodiment, the primer composition includes all of the referenced sets.

In some embodiments, a primer set is selected having a specificity for amplifying a hly gene of L. monocytogenes, or for amplifying a fragment of the hly gene of L. monocytogenes. For example, the primer set selected having a specificity for amplifying a hly gene of L. monocytogenes, or a fragment of the hly gene of L. monocytogenes, may include isolated nucleic acid sequences having SEQ ID NO: 1 and SEQ ID NO: 2, or a nucleic acid molecule with at least 90% sequence homology to said SEQ ID sequences or a labeled derivative thereof.

In some embodiments, a primer set is selected having a specificity for amplifying an iap gene of L. innocua, L. grayi subsp. grayi, and/or L. grayi subsp. murrayi, or for amplifying a fragment of the iap gene. For example, the primer set selected having a specificity for amplifying an iap gene of L. innocua, L. grayi subsp. grayi, and/or L. grayi subsp. murrayi, or a fragment of the iap gene, may include isolated nucleic acid sequences having SEQ ID NO: 3, SEQ ID NO: 4, and/or SEQ ID NO: 5, or a nucleic acid molecule with at least 90% sequence homology to said SEQ ID sequences or a labeled derivative thereof.

In some embodiments, a primer set is selected having a specificity for amplifying an Ise24 gene of L. seeligeri, or for amplifying a fragment of the Ise24 gene of L. seeligeri. For example, the primer set selected having a specificity for amplifying an Ise24 gene of L. seeligeri, or a fragment of the Ise24 gene of L. seeligeri, may include isolated nucleic acid sequences having SEQ ID NO: 6 and SEQ ID NO: 7, or a nucleic acid molecule with at least 90% sequence homology to said SEQ ID sequences or a labeled derivative thereof.

In some embodiments, a primer set is selected having a specificity for amplifying a smcL gene of L. ivanovii, or for amplifying a fragment of the smcL gene of L. ivanovii. For example, the primer set selected having a specificity for amplifying a smcL gene of L. ivanovii, or a fragment of the smcL gene of L. ivanovii, may include isolated nucleic acid sequences having SEQ ID NO: 8 and SEQ ID NO: 9, or a nucleic acid molecule with at least 90% sequence homology to said SEQ ID sequences or a labeled derivative thereof.

In some embodiments, a primer set is selected having a specificity for amplifying a Lwe1801 gene of L. welshimeri, or for amplifying a fragment of the Lwe1801 gene of L. welshimeri. For example, the primer set selected having a specificity for amplifying a Lwe1801 gene of L. welshimeri, or a fragment of the Lwe1801 gene of L. welshimeri, may include isolated nucleic acid sequences having SEQ ID NO: 10 and SEQ ID NO: 11, or a nucleic acid molecule with at least 90% sequence homology to said SEQ ID sequences or a labeled derivative thereof.

In some embodiments, a primer set is selected having a specificity for amplifying a 23S rDNA gene of genus Listeria, or for amplifying a fragment of the 23S rDNA gene of genus Listeria. For example, the primer set selected having a specificity for amplifying a 23S rDNA gene of genus Listeria, or a fragment of the 23S rDNA gene of genus Listeria, may include isolated nucleic acid sequences having SEQ ID NO: 12 and SEQ ID NO: 13, or a nucleic acid molecule with at least 90% sequence homology to said SEQ ID sequences or a labeled derivative thereof.

In a specific embodiment, a primer composition is selected including a plurality of primer sets configured for detecting multiple species or subspecies of Listeria, and genus Listeria. For example, the primer composition can include a first primer set, the first primer set configured for detecting genus Listeria, and a second primer set configured for detecting a species or subspecies of Listeria. For example, the primer composition can include at least a first primer set, the first primer set including a first primer and a second primer, the first primer and the second primer including SEQ ID NO: 12 and SEQ ID NO: 13. The primer set(s) may also include one or more additional primers operable to amplify at least one additional PCR product. For example, the primer composition also includes a second primer set, the second primer set including at least a third primer and a fourth primer, the third primer and the fourth primer selected from SEQ ID NO: 1-SEQ ID NO: 11. The second primer set is operable to detect a first species and/or subspecies of genus Listeria. In some embodiments, the primer composition also includes at least at least a third primer set, the third primer set including at least a fifth primer and a sixth primer, the fifth primer and the sixth primer selected from SEQ ID NO: 1 through SEQ ID NO: 11. The third primer set is operable to detect a second species and/or subspecies of genus Listeria. In a specific embodiment, the primer composition includes five or more primer sets configured for detecting different species and/or subspecies of genus Listeria, and a primer set configured for detecting genus Listeria. For example, the primer composition can include a first primer set including two primers including SEQ ID NO: 12 and SEQ ID NO: 13. The primer composition also includes a second primer set, a third primer set, a fourth primer set, a fifth primer set, and a sixth primer set, each primer set including at least two primers selected from SEQ ID NO: 1-SEQ ID NO: 11, each of the primer sets configured to detect one or more different species or subspecies of Listeria. In some embodiments, each primer set is configured to detect a specific gene corresponding to a species or subspecies of Listeria, or genus Listeria (e.g., as described with reference to FIG. 1).

### Example Kit for Detection of Listeria Species and/or Subspecies, and Genus

A “kit,” as used herein, refers to a combination of at least some items for performing a PCR assay for performing the detection of one or more species and/or subspecies of Listeria and Genus Listeria. Embodiments of kits may include at least one or more of the following reagents: at least one primer set specific for detection of a Listeria species and/or subspecies; at least one primer set specific for detection of genus Listeria; at least one probe (e.g. a TAQMAN™ probe of ThermoFisher Scientific) specific for detection of a Listeria species and/or subspecies, or genus Listeria; an internal positive control DNA to monitor presence of PCR inhibitors from various food and environmental sources; a baseline control; reagents for sample collection; reagents for isolating nucleic acid such as particles; columns; magnetic beads; lysis buffers; wash buffers; elution buffers; proteases; a DNA polymerase or an enzymatically active mutant or variant thereof; a DNA polymerase buffer; deoxyribonucleotides dATP, dCTP, dGTP, or dTTP. In certain kit embodiments, amplification primers are attached to a solid support such as a microarray.

The container means of the kits will generally include at least one vial, test tube, flask, bottle, syringe or other packaging means, into which a component can be placed, and in some embodiments, suitably aliquoted. Where more than one component is included in the kit (they can be packaged together), the kit also will generally contain at least one second, third or other additional container into which the additional components can be separately placed. However, various combinations of components can be packaged in a container means. The kits may also include reagent containers in close confinement for commercial sale. Such containers can include injection or blow-molded plastic containers into which the desired container means are retained. When the components of the kit are provided in one and/or more liquid solutions, the liquid solution includes an aqueous solution that can be a sterile aqueous solution.

In some embodiments, one or more kit component is lyophilized and provided as dried powder(s). For example, primers and TAQMAN® probes may be lyophilized. When reagents and/or components are provided as a dry powder, the powder can be reconstituted by the addition of a suitable solvent. In some embodiments, the solvent is provided in another container means. Kits can also include an additional container means for containing a sterile, pharmaceutically acceptable buffer and/or other diluent.

A kit can also include instructions for employing the kit components as well as the use of any other reagent not included in the kit. Instructions can include variations that can be implemented.

An exemplary kit includes a primer composition for detecting one or more Listeria species, subspecies, or strain. For example, the primer composition can include at least one primer pair having specificity for amplifying a gene of a specific Listeria species including, but not necessarily limited to: a hly gene, an iap gene, an Ise24 gene, a smcL gene, a Lwe1801, and so forth or for amplifying a fragment of the gene of Listeria species, subspecies, or strain.

In some embodiments, a kit may be configured for detection L. monocytogenes. For example, the kit may include a primer set having a specificity for amplifying a hly gene of L. monocytogenes, or for amplifying a fragment of the hly gene of L. monocytogenes. For example, the primer set having a specificity for amplifying a hly gene of L. monocytogenes, or a fragment of the hly gene of L. monocytogenes, may include isolated nucleic acid sequences having SEQ ID NO: 1 and SEQ ID NO: 2, or a nucleic acid molecule with at least 90% sequence homology to said SEQ ID sequences or a labeled derivative thereof.

In some embodiments, a kit may be configured for detection. innocua, L. grayi subsp. grayi, and/or L. grayi subsp. murrayi. For example, the kit may include a primer set having a specificity for amplifying an iap gene of L. innocua, L. grayi subsp. grayi, and/or L. grayi subsp. murrayi, or for amplifying a fragment of the iap gene. For example, the primer set having a specificity for amplifying an iap gene of L. innocua, L. grayi subsp. grayi, and/or L. grayi subsp. murrayi, or a fragment of the iap gene, may include isolated nucleic acid sequences having SEQ ID NO: 3, SEQ ID NO: 4, and/or SEQ ID NO: 5, or a nucleic acid molecule with at least 90% sequence homology to said SEQ ID sequences or a labeled derivative thereof.

In some embodiments, a kit may be configured for detection L. seeligeri. For example, the kit may include a primer set having a specificity for amplifying an Ise24 gene of L. seeligeri, or for amplifying a fragment of the Ise24 gene of L. seeligeri. For example, the primer set having a specificity for amplifying an Ise24 gene of L. seeligeri, or a fragment of the Ise24 gene of L. seeligeri, may include isolated nucleic acid sequences having SEQ ID NO: 6 and SEQ ID NO: 7, or a nucleic acid molecule with at least 90% sequence homology to said SEQ ID sequences or a labeled derivative thereof.

In some embodiments, a kit may be configured for detection L. ivanovii. For example, the kit may include a primer set having a specificity for amplifying a smcL gene of L. ivanovii, or for amplifying a fragment of the smcL gene of L. ivanovii. For example, the primer set having a specificity for amplifying a smcL gene of L. ivanovii, or a fragment of the smcL gene of L. ivanovii, may include isolated nucleic acid sequences having SEQ ID NO: 8 and SEQ ID NO: 9, or a nucleic acid molecule with at least 90% sequence homology to said SEQ ID sequences or a labeled derivative thereof.

In some embodiments, a kit may be configured for detection L. welshimeri. For example, the kit may include a primer set having a specificity for amplifying a Lwe1801 gene of L. welshimeri, or for amplifying a fragment of the Lwe1801 gene of L. welshimeri. For example, the primer set having a specificity for amplifying a Lwe1801 gene of L. welshimeri, or a fragment of the Lwe1801 gene of L. welshimeri, may include isolated nucleic acid sequences having SEQ ID NO: 10 and SEQ ID NO: 11, or a nucleic acid molecule with at least 90% sequence homology to said SEQ ID sequences or a labeled derivative thereof.

In embodiments, the kit is also configured for detection genus Listeria. For example, the kit may include a primer set having a specificity for amplifying a 23S rDNA gene of genus Listeria, or for amplifying a fragment of the 23S rDNA gene of genus Listeria. For example, the primer set having a specificity for amplifying a 23S rDNA gene of genus Listeria, or a fragment of the 23S rDNA gene of genus Listeria, may include isolated nucleic acid sequences having SEQ ID NO: 10 and SEQ ID NO: 11, or a nucleic acid molecule with at least 90% sequence homology to said SEQ ID sequences or a labeled derivative thereof.

In a specific embodiment, the kit includes a primer composition including a plurality of primer sets configured for detecting multiple species or subspecies of Listeria, and genus Listeria. For example, the primer composition can include a first primer set, the first primer set configured for detecting genus Listeria, and a second primer set configured for detecting a species and/or subspecies of Listeria. For example, the primer composition can include at least a first primer set, the first primer set including a first primer and a second primer. The first primer and the second primer can include SEQ ID NO: 12 and SEQ ID NO: 13, respectively. The first primer set is operable to detect the genus Listeria. The primer set(s) may also include one or more additional primers operable to amplify at least one additional PCR product. For example, the primer composition also includes at least a second primer set, the second primer set including at least a third primer and a fourth primer, the third primer and the fourth primer selected from SEQ ID NO: 1-SEQ ID NO: 11. The second primer set is operable to detect a first species and/or subspecies of genus Listeria. In some embodiments, the primer composition also includes at least at least a third primer set, the third primer set including at least a fifth primer and a sixth primer, the fifth primer and the sixth primer selected from SEQ ID NO: 1 through SEQ ID NO: 11. The third primer set is operable to detect a second species and/or subspecies of genus Listeria. In a specific embodiment, the primer composition includes five or more primer sets configured for detecting different species and/or subspecies of genus Listeria, and a primer set configured for detecting the genus Listeria. For example, the primer composition can include a first primer set including two primers. The two primers can include SEQ ID NO: 12 and SEQ ID NO: 13. The primer composition also includes a second primer set, a third primer set, a fourth primer set, a fifth primer set, and a sixth primer set, each primer set including at least two primers selected from SEQ ID NO: 1-SEQ ID NO: 11, each of the primer sets configured to detect one or more different species or subspecies of Listeria. In some embodiments, each primer set is configured to detect a specific gene corresponding to a species or subspecies of Listeria, or genus Listeria (e.g., as described with reference to FIG. 1).

### Example Detection of Listeria Species and/or Subspecies, and Genus

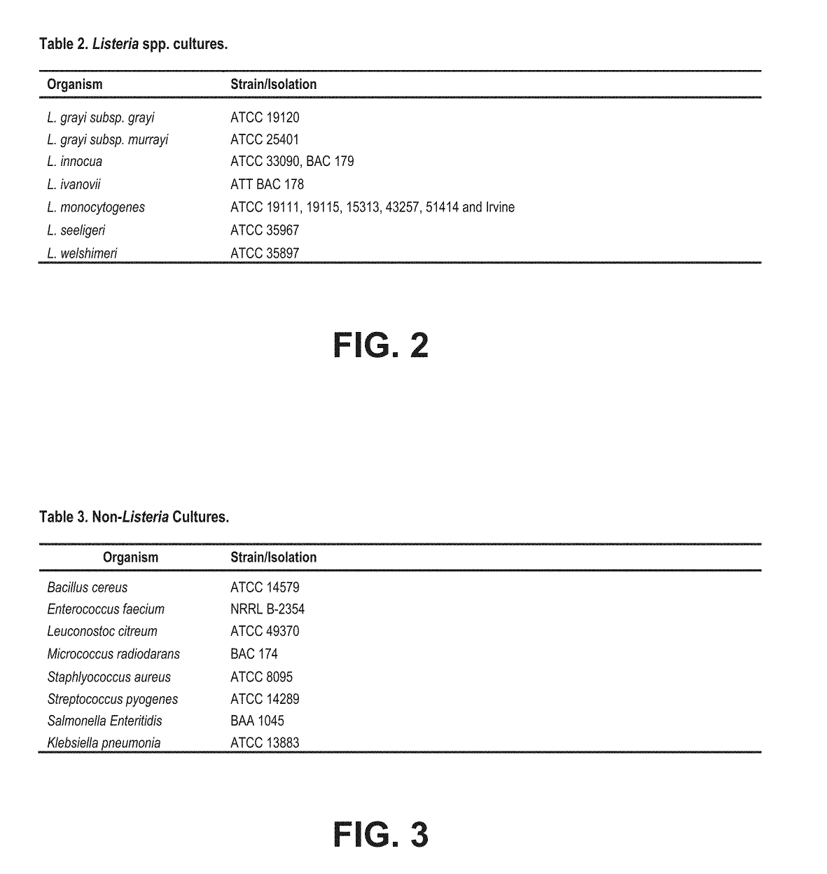

Strains used for the development (e.g., as described with reference to FIGS. 2 and 3) of the multiplex PCR methods were started for propagation from freezer stocks using BHI broth and streaked to Bio-Rad Rapid L. mono agar (Listeria spp. strains) or TSA agar (non-Listeria strains) prior to testing. For all Listeria spp. ATCC strains, the biochemical characteristics of each strain grown on the Bio-Rad RAPID L. mono agar were observed to determine the presumptive species identity. L. monocytogenes colonies form characteristic blue colony without a yellow halo and the other Listeria spp. colonies are white with or without a yellow halo.

Multiplex PCR Optimization.

The QIAGEN DNA extraction/purification procedure for gram-positive bacteria was performed on a loopful of isolated growth on the Bio-Rad RAPID L. mono agar for all of the Listeria spp. ATCC strains. The purified genomic DNA was amplified during PCR by using species and genus-specific primers for Listeria on the Bio-Rad C1000 thermal cycler.

Multiple primer pairs were combined in a primer composition. All the primers (100 μM stock concentration) designated for each method were combined with TE buffer to make the primer mix (total volume of 500 μl) for the multiplex PCR reactions. Six species-specific primer sets targeting hly, iap, lse24, smcL, Lwe1801, and one genus-specific primer set targeting 23S rRNA were used for substantially simultaneous differentiation of L. monocytogenes, L. innocua, L. grayi subsp. grayi, L. grayi subsp. murrayi, L. seeligeri, L. ivanovii, L. welshimeri, and the genus Listeria, respectively, as illustrated in FIG. 2. The PCR primers (e.g., as described with reference to FIG. 1) were selected based on amplification of a single PCR product (e.g., a nucleic acid, a fragment thereof, or a complement thereof) at the expected size and absence of masking effects on the other primers included in each primer mix. Primer-Blast analysis confirmed the expected PCR product sizes for all primer sets used within each method. Multiplex PCR was prepared in 50 μl reaction mixtures containing: 25 μl of QIAGEN multiplex PCR master mix, 5 μl of primer mix, 15 μl nanopure water, and 5 μl of purified DNA.

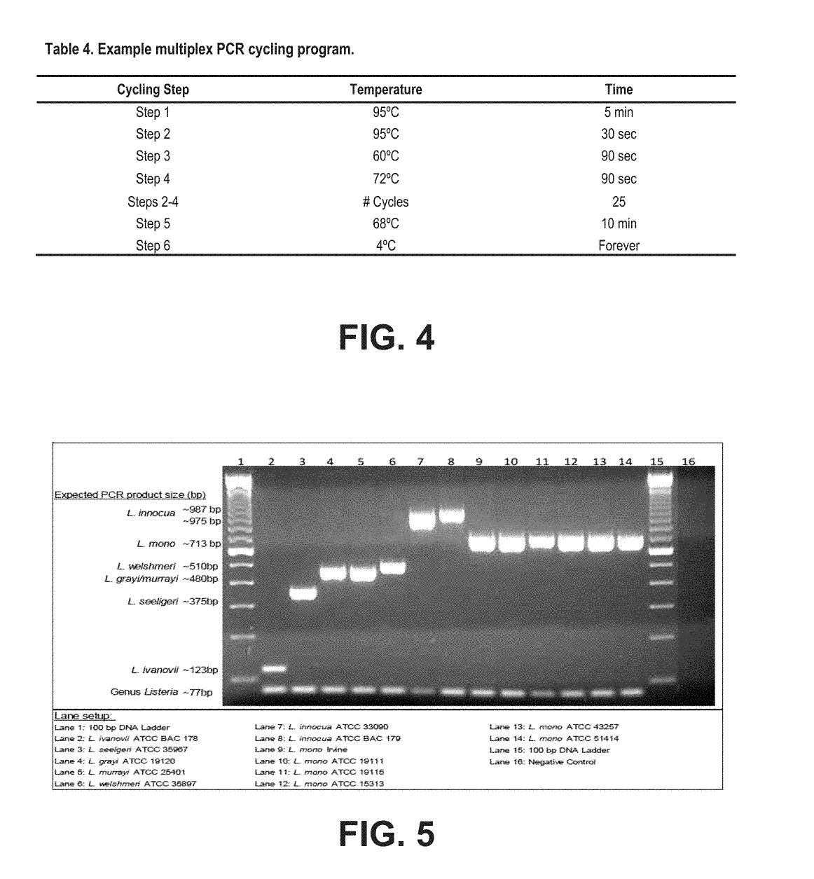

Gradient PCR tests were performed and determined the annealing temperature of approximately 60° C. was optimal for the cycling program for both multiplex PCR methods. The optimized multiplex PCR cycling program was performed on all of the Listeria spp. ATCC isolates to create PCR product to identify the species (e.g., as described with reference to FIG. 4). The optimized PCR cycling program includes heating the sample at a temperature of approximately 95° C. for approximately 5 minutes to activate the DNA in the polymerase. The sample is then heated at a temperature of approximately 95° C. for approximately 30 seconds to denature the target DNA in the sample to form single stranded target DNA. The sample is then annealed at temperature of approximately 60° C. for approximately 90 seconds to hybridize the target DNA to the primers. The sample is then heated at a temperature of approximately 72° C. to elongate the hybridized DNA. The denaturation, annealing, and elongation steps can be repeated to ensure adequate denaturation, annealing, and elongation. In some implementations, these steps can be repeated 20 to 30 times. Then, the sample is heated to a temperature of approximately 68° C. This step is optionally completed to ensure that any remaining single stranded DNA is elongated. The resulting PCR product is then cooled indefinitely. For example, the PCR product can be cooled and stored at a temperature of approximately 4° C.

Identification Analysis.

DNA extracted from 13 Listeria spp. ATCC strains and 64 unknown presumptive Listeria spp. isolates were tested by the multiplex PCR method to evaluate the efficiency of the selected primer sets. Agarose gel analysis was performed on the PCR products generated by the multiplex PCR method. The multiplex PCR method can generate at least one species-specific PCR product and at least one genus-specific PCR product per test. This allows substantially simultaneous detection of both species and genus for Listeria identification. The PCR products for each strain were loaded into a 2% agarose gel and ran at 150 volts for 90 minutes during gel electrophoresis. Species identification was determined based on the specific size (bp; migration position on a gel) of the PCR product during the agarose gel analysis (e.g., as described with reference to FIG. 1).

Exclusivity Testing.

The QIAGEN DNA extraction/purification procedures for gram-positive and gram-negative bacteria were performed on a loopful of isolated growth on TSA agar for all of the non-Listeria strains (e.g., as described with reference to FIG. 3). The purified genomic DNA was tested by the multiplex PCR method to verify the specificity. For specificity verification, no substantial amplification of PCR products should be observed after testing all of the non-Listeria strains by the multiplex PCR method.

Verification of Multiplex PCR Method.

Unknown Listeria spp. isolates collected were streaked onto Bio-Rad RAPID L. mono agar. QIAGEN DNA extraction/purification procedure for gram-positive bacteria was performed on a loopful of isolated growth of each isolate. The purified genomic DNA was tested by the multiplex PCR method to determine the specific species of each isolate.

Results.

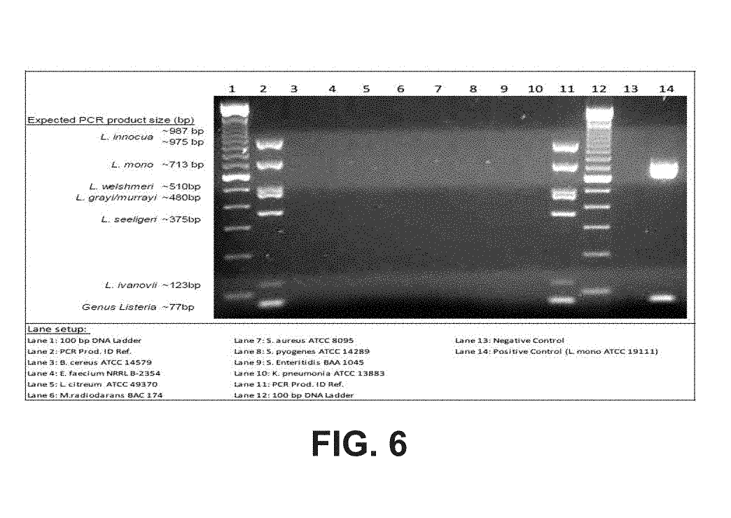

FIG. 5 illustrates an agarose gel analysis showing the approximate sizes (e.g., migration position on the gel) of both species and genus PCR products for each Listeria sp. ATTC strain during optimization of the multiplex PCR method. The multiplex PCR method substantially simultaneously amplified both a species-specific and a genus-specific PCR product per strain and successfully identified all 13 ATCC strains of Listeria. Specificity was verified by testing non-Listeria strains and observing no substantial amplification of PCR products. The agarose gel analysis shows no PCR products that were substantially amplified by the multiplex PCR method after testing the non-Listeria strains (e.g., as described with reference to FIG. 6). This confirmed the high specificity of the designed primer sets for each Listeria sp. Additionally, this method identified 22 unknown isolates as L. monocytogenes (hly ˜713 bp), 42 isolates as L. innocua (iap ˜975-987 bp), and as expected demonstrated that all 64 isolates were within the genus Listeria (23S rRNA ˜77 bp).

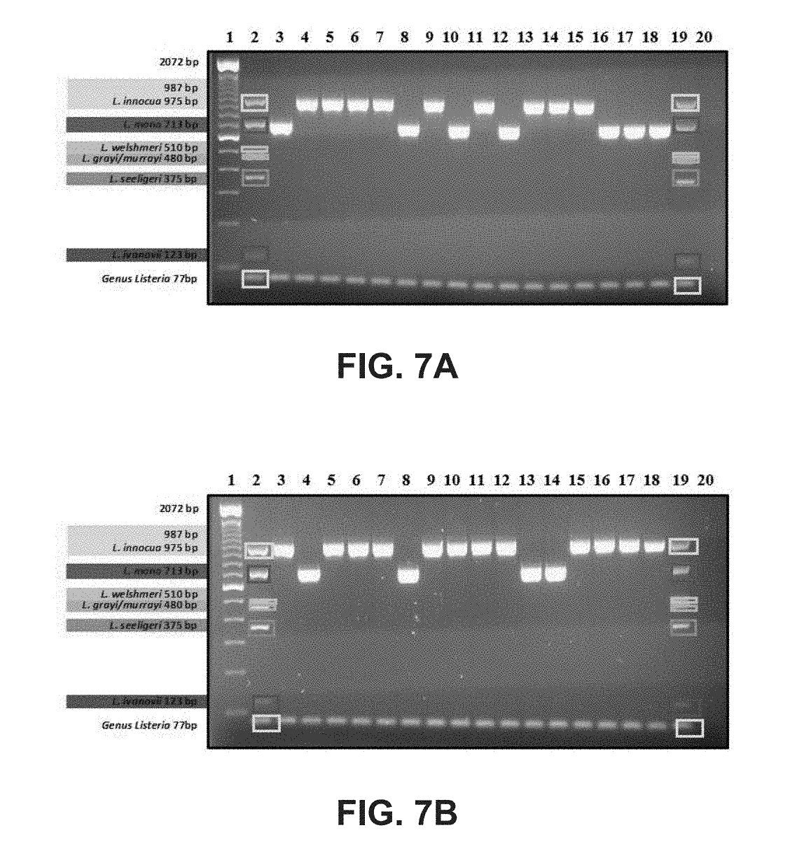

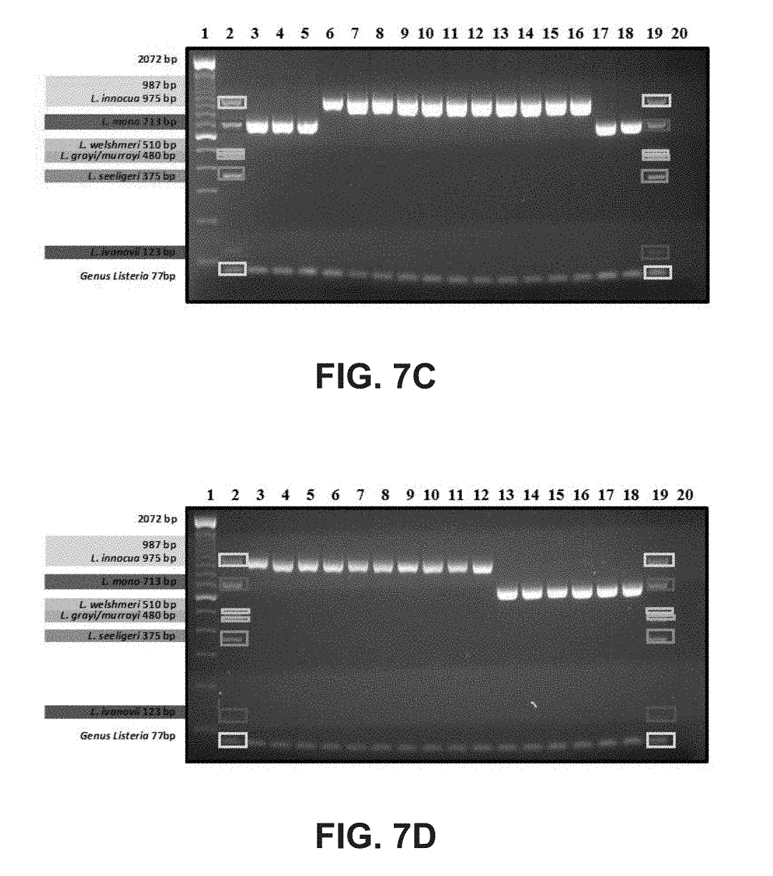

Overall, the multiplex PCR method was efficient and 100% accurate for species-specific identification of Listeria. FIGS. 7A through 7D illustrate a series of agarose gels that show both the species and genus PCR products for the 64 unknown Listeria spp. Isolates amplified by the multiplex PCR method after completing the verification testing. PCR product reference ladders (lanes 2 and 19) show the migration positions of all the species or genus-specific PCR product sizes during gel electrophoresis, mimicking a DNA ladder with multiple bands at different sizes. Still referring to FIGS. 7A through 7D, the multiplex PCR method produced a band for each species of Listeria: L. innocua (975-987 bp), L. monocytogenes (713 bp), L. welshmeri (510 bp), L. grayi/murrayi (480 bp), L. seeligeri (375 bp), and L. ivanovii (123 bp). The multiplex PCR method also produced a band for genus Listeria (77 bp).

This multiplex PCR method can offer rapid identification of presumptive Listeria spp. isolates. This knowledge can enhance risk assessments and provide information regarding the prevalence and potential co-existence of each species of Listeria in food products and the food production environment. This method may also be used as the initial detection method for environmental samples.

It is to be understood that the embodiments described herein can be implemented by various types of electro-mechanical systems having a wide range of electrical components including, but not necessarily limited to: hardware, software, firmware, and/or virtually any combination thereof. For example, an automated analysis system can include a computing device including a processor and a memory. The processor provides processing functionality for the computing device and may include any number of processors, micro-controllers, or other processing systems, and resident or external memory for storing data and other information accessed or generated by the computing device. The processor may execute one or more software programs that implement the techniques and modules described herein. The processor is not limited by the materials from which it is formed or the processing mechanisms employed therein and, as such, may be implemented via semiconductor(s) and/or transistors (e.g., electronic integrated circuits (ICs)), and so forth.

The memory is an example of device-readable storage media that provides storage functionality to store various data associated with the operation of the computing device, such as the software program and code segments mentioned above, or other data to instruct the processor and other elements of the computing device to perform the techniques described herein. Although a single memory is mentioned above, a wide variety of types and combinations of memory may be employed. The memory may be integral with the processor, stand-alone memory, or a combination of both. The memory may include, for example, removable and non-removable memory elements such as RAM, ROM, Flash (e.g., SD Card, mini-SD card, micro-SD Card), magnetic, optical, USB memory devices, and so forth. In embodiments of the computing device, the memory may include removable ICC (Integrated Circuit Card) memory such as provided by SIM (Subscriber Identity Module) cards, USIM (Universal Subscriber Identity Module) cards, UICC (Universal Integrated Circuit Cards), and so on.

The computing device includes a display to display information to a user of the computing device. In embodiments, the display may include a CRT (Cathode Ray Tube) display, an LED (Light Emitting Diode) display, an OLED (Organic LED) display, an LCD (Liquid Crystal Diode) display, a TFT (Thin Film Transistor) LCD display, an LEP (Light Emitting Polymer) or PLED (Polymer Light Emitting Diode) display, and so forth, configured to display text and/or graphical information such as a graphical user interface. The display may be backlit via a backlight such that it may be viewed in the dark or other low-light environments.

The display may be provided with a touch screen to receive input (e.g., data, commands, etc.) from a user. For example, a user may operate the computing device by touching the touch screen and/or by performing gestures on the touch screen. In some embodiments, the touch screen may be a capacitive touch screen, a resistive touch screen, an infrared touch screen, combinations thereof, and the like. The computing device may further include one or more input/output (I/O) devices (e.g., a keypad, buttons, a wireless input device, a thumbwheel input device, a trackstick input device, and so on). The I/O devices may include one or more audio I/O devices, such as a microphone, speakers, and so on.

The computing device may also include a communication module representative of communication functionality to permit computing device to send/receive data between different devices (e.g., components/peripherals) and/or over one or more networks. Communication module may be representative of a variety of communication components and functionality including, but not necessarily limited to: a browser; a transmitter and/or receiver; data ports; software interfaces and drivers; networking interfaces; data processing components; and so forth.

The one or more networks are representative of a variety of different communication pathways and network connections which may be employed, individually or in combinations, to communicate among the components of the system. Thus, the one or more networks may be representative of communication pathways achieved using a single network or multiple networks. Further, the one or more networks are representative of a variety of different types of networks and connections that are contemplated including, but not necessarily limited to: the Internet; an intranet; a Personal Area Network (PAN); a Local Area Network (LAN) (e.g., Ethernet); a Wide Area Network (WAN); a satellite network; a cellular network; a mobile data network; wired and/or wireless connections; and so forth.

Examples of wireless networks include, but are not necessarily limited to: networks configured for communications according to: one or more standard of the Institute of Electrical and Electronics Engineers (IEEE), such as 802.11 or 802.16 (Wi-Max) standards; Wi-Fi standards promulgated by the Wi-Fi Alliance; Bluetooth standards promulgated by the Bluetooth Special Interest Group; and so on. Wired communications are also contemplated such as through Universal Serial Bus (USB), Ethernet, serial connections, and so forth.

The computing device is described as including a user interface, which is storable in memory and executable by the processor. The user interface is representative of functionality to control the display of information and data to the user of the computing device via the display. In some implementations, the display may not be integrated into the computing device and may instead be connected externally using universal serial bus (USB), Ethernet, serial connections, and so forth. The user interface may provide functionality to allow the user to interact with one or more applications of the computing device by providing inputs (e.g., sample identities, PCR cycling protocols, et.) via the touch screen and/or the I/O devices. For example, the user interface may cause an application programming interface (API) to be generated to expose functionality to a temperature control module to configure the application for display by the display or in combination with another display. In embodiments, the API may further expose functionality to configure an inline amplification control module, an inline detection control module, or a combination thereof, to allow the user to interact with an application by providing inputs via the touch screen and/or the I/O devices to provide desired PCR parameters for analysis.

The inline amplification control module and/or the inline detection control module may include software, which is storable in memory and executable by the processor, to perform a specific operation or group of operations to furnish functionality to the computing device. The inline amplification control module provides functionality to control the amplification of, for example, a DNA sample and/or the samples from the automated analysis system. For example, the inline amplification control module may control the temperature of each PCR cycle. The detection control module provides functionality to control detection of, for example, one or more organisms in a sample. For example, the detection control module may control one more automated analysis processes (e.g., automated gel electrophoresis).

In implementations, the user interface may include a browser (e.g., for implementing functionality of the control modules described herein). The browser enables the computing device to display and interact with content such as a webpage within the World Wide Web, a webpage provided by a web server in a private network, and so forth. The browser may be configured in a variety of ways. For example, the browser may be configured as an amplification control module or detection control module accessed by the user interface. The browser may be a web browser suitable for use by a full resource device with substantial memory and processor resources (e.g., a smart phone, a personal digital assistant (PDA), etc.).

Generally, any of the functions described herein can be implemented using software, firmware, hardware (e.g., fixed logic circuitry), manual processing, or a combination of these implementations. The terms “module” and “functionality” as used herein generally represent software, firmware, hardware, or a combination thereof. The communication between modules in the system, for example, can be wired, wireless, or some combination thereof. In the case of a software implementation, for instance, a module may represent executable instructions that perform specified tasks when executed on a processor, such as the processor described herein. The program code can be stored in one or more device-readable storage media, an example of which is the memory associated with the computing device.

It is to be understood that embodiments of the present invention described above are intended to be merely exemplary. Those skilled in the art will recognize, or be able to ascertain using no more than routine experimentation, numerous equivalents to the specific procedures described herein. All such equivalents are considered to be within the scope of the present invention and are covered by the following claims.

It is further contemplated that any embodiment or implementation of the disclosure manifested above as a system or method may include at least a portion of any other embodiment or implementation described herein. Those having skill in the art will appreciate that there are various embodiments or implementations by which systems and methods described herein can be implemented, and that the implementation will vary with the context in which an embodiment of the disclosure is deployed.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A primer composition comprising:
a first primer set operable to amplify a first nucleic acid, a fragment thereof, a complement thereof, or a gene, the first primer set including at least a first primer sequence and a second primer sequence, the at least a first primer sequence and a second primer sequence specific for genus Listeria;
a second primer set operable to amplify at least a second nucleic acid, a fragment thereof, or a complement thereof, or a gene, the second primer set including at least a third primer sequence and a fourth primer sequence, the at least a third primer sequence and a fourth primer sequence specific for at least one of a first species of Listeria or a subspecies thereof; and
at least a third primer set operable to amplify at least a third nucleic acid, a fragment thereof, or a complement thereof, or a gene, the at least a third primer set including at least a fifth primer sequence and a sixth primer sequence, the at least a fifth primer sequence and a sixth primer sequence specific for at least one of a second species of Listeria or a subspecies thereof.

2. The primer composition of claim 1, wherein each of the at least a first primer sequence and at least a second primer sequence comprise at least one of a nucleic acid molecule having a sequence comprised in SEQ ID NO: 12 and SEQ ID NO: 13, a nucleic acid molecule with at least 90% sequence homology thereto, or a nucleic acid molecule complementary thereto.

3. The primer composition of claim 1, wherein each of the at least a third primer sequence and at least a fourth primer sequence comprise at least one of a nucleic acid molecule having a sequence comprised in SEQ ID NO: 1 through SEQ ID NO: 11, a nucleic acid molecule with at least 90% sequence homology thereto, or a nucleic acid molecule complementary thereto.

4. The primer composition of claim 1, wherein each of the at least a fifth primer sequence and at least a sixth primer sequence comprise at least one of a nucleic acid molecule having a sequence comprised in SEQ ID NO: 1 through SEQ ID NO: 11, a nucleic acid molecule with at least 90% sequence homology thereto, or a nucleic acid molecule complementary thereto.

5. The primer composition of claim 1, wherein the at least a third primer sequence and a fourth primer sequence comprise at least one of a set of sequences comprised of at least one of SEQ ID NO: 1 and SEQ ID NO: 2, SEQ ID NO: 3 through SEQ ID NO: 5, SEQ ID NO: 6 and SEQ ID NO: 7, SEQ ID NO: 8 and SEQ ID NO: 9, or SEQ ID NO: 10 and SEQ ID NO: 11, a set of nucleic acid molecules with at least 90% sequence homology to the set of sequences, a set of nucleic acid molecules complementary to the set of sequences, or a set of nucleic acid molecules complementary to the nucleic acid molecules with at least 90% sequence homology to the set of sequences.

6. The primer composition of claim 1, wherein the at least a third primer sequence and a fourth primer sequence are specific for at least one of L. monocytogenes, L. innocua, L. ivanovii, L. seeligeri, L. welshimeri, or L. grayi.

7. A method for determining the presence of Listeria organism in a sample, the method comprising:
contacting the sample with a primer composition, the primer composition including:
a first primer set operable to amplify a first nucleic acid, a fragment thereof, a complement thereof, or a gene;
at least one additional primer set operable to amplify at least a second nucleic acid, a fragment thereof, a complement thereof, or a gene;
detecting at least one of the first amplified nucleic acid, a fragment thereof, a complement thereof, or a gene, and the at least a second amplified nucleic acid, a fragment thereof, a complement thereof, or a gene, wherein detecting the first amplified nucleic acid, a fragment thereof, a complement thereof, or a gene is indicative of the presence of genus Listeria in the sample, and detecting the at least a second amplified nucleic acid, a fragment thereof, a complement thereof, or a gene is indicative of at least one of a species of Listeria or a subspecies thereof in the sample.

8. The method for determining the presence of Listeria organism in a sample of claim 7, wherein the first primer set comprises at least one of a pair of nucleic acid molecules having the sequences comprised in SEQ ID NO: 12 and SEQ ID NO: 13, a pair of nucleic acid molecules with at least 90% sequence homology thereto, or a pair of nucleic acid molecules complementary thereto.

9. The method for determining the presence of Listeria organism in a sample of claim 7, wherein the at least one additional primer set comprises at least one of a pair of nucleic acid molecules having sequences comprised in SEQ ID NO: 1 through SEQ ID NO: 11, a pair of nucleic acid molecules with at least 90% sequence homology thereto, or a pair of nucleic acid molecules complementary thereto

10. The method for determining the presence of Listeria organism in a sample of claim 7, wherein the at least one additional primer set is operable to amplify at least a third nucleic acid, a fragment thereof, a complement thereof, or a gene.

11. The method for determining the presence of Listeria organism in a sample of claim 10, further comprising detecting the at least a third amplified nucleic acid, fragment thereof, or complement thereof, wherein detecting the at least a third amplified nucleic acid, a fragment thereof, a complement thereof, or a gene is indicative of at least one of an additional species of Listeria or an additional subspecies of Listeria.

12. The method for determining the presence of Listeria organism in a sample of claim 7, wherein detecting the at least a second amplified nucleic acid, a fragment thereof, a complement thereof, or a gene is indicative of the presence of at least one of L. monocytogenes, L. innocua, L. ivanovii, L. seeligeri, L. welshimeri, or L. grayi in the sample.

13. The method for determining the presence of Listeria organism in a sample of claim 7, wherein the detecting is performed by a PCR technique.

14. The method for determining the presence of Listeria organism in a sample of claim 13, wherein the PCR technique comprises at least one of agarose gel assay or a real-time PCR technique.

15. The method for determining the presence of Listeria organism in a sample of claim 7, further comprising subjecting the sample to a series of PCR cycles configured to anneal the at least a first primer set to the at least a first nucleic acid, a fragment thereof, a complement thereof, or a gene and to anneal the at least a second primer set to the at least a second nucleic acid, a fragment thereof, a complement thereof, or a gene.

16. A primer composition comprising:
a first primer set operable to amplify a first nucleic acid, a fragment thereof, a complement thereof, or a gene, the first primer set comprising at least one of a first set of sequences comprised of SEQ ID NO: 12 and SEQ ID NO: 13, a set of nucleic acid molecules with at least 90% sequence homology to the first set of sequences, a set of nucleic acid molecules complementary to the first set of sequences, or a set of nucleic acid molecules complementary to the nucleic acid molecules with at least 90% sequence homology to the first set of sequences; and
at least one additional primer set operable to amplify at least a second nucleic acid, a fragment thereof, a complement thereof, or a gene, the at least one additional primer set comprising at least one of a second set of sequences comprised of at least one of SEQ ID NO: 1 and SEQ ID NO: 2, SEQ ID NO: 3 through SEQ ID NO: 5, SEQ ID NO: 6 and SEQ ID NO: 7, SEQ ID NO: 8 and SEQ ID NO: 9, or SEQ ID NO: 10 and SEQ ID NO: 11, a set of nucleic acid molecules with at least 90% sequence homology to the second set of sequences, a set of nucleic acid molecules complementary to the second set of sequences, or a set of nucleic acid molecules complementary to the nucleic acid molecules with at least 90% sequence homology to the second set of sequences.

17. The primer composition of claim 16, wherein the first primer set is specific for genus Listeria.

18. The primer composition of claim 16, wherein the at least one additional primer set is specific for at least one of L. monocytogenes, L. innocua, L. ivanovii, L. seeligeri, L. welshimeri, or L. grayi.

19. The primer composition of claim 16, wherein the at least one additional primer set further comprises:
a second primer set, the second primer set operable to amplify the at least a second nucleic acid, a fragment thereof, a complement thereof, or a gene, the second primer set comprising at least one of a second set of sequences comprised of at least one of SEQ ID NO: 1 and SEQ ID NO: 2, SEQ ID NO: 3 through SEQ ID NO: 5, SEQ ID NO: 6 and SEQ ID NO: 7, SEQ ID NO: 8 and SEQ ID NO: 9, or SEQ ID NO: 10 and SEQ ID NO: 11, a set of nucleic acid molecules with at least 90% sequence homology to the second set of sequences, a set of nucleic acid molecules complementary to the second set of sequences, or a set of nucleic acid molecules complementary to the nucleic acid molecules with at least 90% sequence homology to the second set of sequences; and
a third primer set, the third primer set operable to amplify at least a third nucleic acid, a fragment thereof, a complement thereof, or a gene, the third primer set comprising at least one of a third set of sequences different than the second set of sequences, the third set of sequences comprised of at least one of SEQ ID NO: 1 and SEQ ID NO: 2, SEQ ID NO: 3 through SEQ ID NO: 5, SEQ ID NO: 6 and SEQ ID NO: 7, SEQ ID NO: 8 and SEQ ID NO: 9, or SEQ ID NO: 10 and SEQ ID NO: 11, a set of nucleic acid molecules with at least 90% sequence homology to the third set of sequences, a set of nucleic acid molecules complementary to the third set of sequences, or a set of nucleic acid molecules complementary to the nucleic acid molecules with at least 90% sequence homology to the third set of sequences.

20. The primer composition of claim 16, wherein the second primer set is specific for a first species of Listeria or subspecies of thereof, and the third primer set is specific for a second species of Listeria or subspecies of thereof.


SEQUENCE LISTING (13 sequences)

SEQ ID NO: 1 - DNA, 20 bp, Listeria monocytogenes
tccgcctgcaagtcctaaga

SEQ ID NO: 2 - DNA, 20 bp, Listeria monocytogenes
gcgcttgcaactgctcttta

SEQ ID NO: 3 - DNA, 22 bp, Listeria innocua
actagcactccagttgttaaac

SEQ ID NO: 4 - DNA, 21 bp, Listeria grayi and Listeria murrayi
ccagcagtttctaaacctgct

SEQ ID NO: 5 - DNA, 21 bp, Listeria innocua, Listeria grayi, Listeria murrayi
ttatacgcgaccgaagccaac

SEQ ID NO: 6 - DNA, 20 bp, Listeria seeligeri
cgcgacggctaaagtactaa

SEQ ID NO: 7 - DNA, 20 bp, Listeria seeligeri
attgctcgctttgaagtcgt

SEQ ID NO: 8 - DNA, 22 bp, Listeria ivanovii
acccgtaattgggagtaaacat

SEQ ID NO: 9 - DNA, 23 bp, Listeria ivanovii
tgttggcttttttcgagaatagg

SEQ ID NO: 10 - DNA, 20 bp, Listeria welshimeri
ccacctcattcgtgttttca

SEQ ID NO: 11 - DNA, 20 bp, Listeria welshimeri
ttggcactgagcagatagga

SEQ ID NO: 12 - DNA, 20 bp, Listeria
aggatagggaatcgcacgaa

SEQ ID NO: 13 - DNA, 18 bp, Listeria
ttcgcgagaagcggattt